(19) United States
(12) Patent Application Publication
(10) Pub. No.: US 2010/0048996 A1
(43) Pub. Date: Feb. 25, 2010

(54) Medical Instrument Insertion Guide System

(75) Inventor: Satoshi Makiyama, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/543,808

(22) Filed: Aug. 19, 2009

(30) Foreign Application Priority Data
Aug. 22, 2008 (JP) 2008214161

(51) Int. Cl. A61B 1/00

(52) U.S. Cl. 600/114

(57) ABSTRACT

A medical instrument insertion guide system according to the present invention includes: a medical instrument including an insertion portion to be inserted into a body cavity; a double flexible bag member that is an annular member made of flexible material and supplied with a gas and then sealed, and includes an inner surface and an outer surface to be continuously moved in a longitudinal axis direction; and a pressing member that intermittently presses the inner surface near a rear end surface in the axial direction of the double flexible bag member, and turns outward and advances a distal end surface of the double flexible bag member, wherein the pressing member introduces the insertion portion of the medical instrument into the body cavity using the longitudinal axis direction of the double flexible bag member as a guide.

This application claims benefit of Japanese Application No. 2008-214161 filed in Japan on Aug. 22, 2008, the contents of which are incorporated by this reference.

### BACKGROUND OF THE INVENTION

1. Field of the Invention

The present invention relates to a medical instrument insertion guide system configured to previously insert a double flexible bag member into a body cavity, and insert an insertion portion of a medical instrument into the body cavity using the double flexible bag member as a guide.

2. Description of the Related Art

Generally, when an insertion portion provided in a medical instrument such as an endoscope or a probe is inserted into a body cavity such as a large intestine or a small intestine in a living body, insertion resistance increases as insertion length increases because the body cavity into which the medical instrument is inserted is elongated and bent.

The insertion portion is fed forward into a subject from outside and advanced in a body cavity such as a large intestine or a small intestine by an operator. To guide a distal end portion of the insertion portion to a target area, an operator needs skills and experiences for performing an appropriate insertion operation while grasping a shape of an intestine or an insertion state of the endoscope in an advancing process of the insertion portion, and the operation is difficult for an inexperienced operator.

Thus, various techniques have been proposed of inserting a flexible guide tube before inserting an insertion portion provided in a medical instrument such as an endoscope or a probe into a body cavity, and inserting the insertion portion of the medical instrument using the guide tube as a guide, thereby reducing load on an operator.

For example, U.S. Pat. No. 5,045,070 discloses a technique of inserting and advancing an insertion portion of a probe or an endoscope into a body cavity using a flexible tube having an inner wall and an outer wall as a guide. As shown in FIGS. 2 and 3 of the publication, the flexible tube is inserted and advanced into the body cavity while being elastically deformably displaced. Alternatively, as shown in FIGS. 4 to 6 of the publication, a technique is disclosed of inserting and advancing the flexible tube by securing one point of the flexible tube to an insertion member, and pressing out and turning outward the inner wall of the flexible tube toward a distal end to form an outer wall.

Further, in FIG. 7 of the publication, a technique is disclosed of inserting and advancing the flexible tube into the body cavity by securing an insertion device on a rear end of the outer wall of the flexible tube, turning inward the distal end of the flexible tube toward the rear end to form an inner wall, and applying pressure to a sealed space formed between the inner wall and the outer wall with a plunger provided in the insertion device to turn the inner wall outward.

Japanese Patent Application Laid-Open Publication No. 2006-523513 discloses a technique of mounting an annular member self-propelled by power to a distal end portion of a medical instrument inserted into a body cavity, and advancing the medical instrument by circulation motion of the annular member.

Further, U.S. Pat. No. 4,871,358 discloses a technique of turning one flexible tube inward to form an inner wall, protruding a free end of the inner wall rearward of a rear end of an outer wall, also securing a grasping portion on the rear end of the outer wall, pressing the free end of the inner wall while grasping the grasping portion, and thus continuously turning outward the inner wall on a distal end side to advance the flexible tube in a longitudinal axis direction.

The present invention has an object to provide a medical instrument insertion guide system that allows easy advance along an inner shape of a body cavity without grasping the inner shape, can easily guide an insertion portion of a medical instrument inserted afterward, can relatively freely move a distal end of the insertion portion, and can provide satisfactory operability of the medical instrument.

### SUMMARY OF THE INVENTION

The present invention provides a medical instrument insertion guide system including: a medical instrument including a long insertion portion to be inserted into a body cavity; a double flexible bag member that is an annular member made of flexible material and supplied with a fluid and then sealed, and includes an inner surface and an outer surface to be continuously moved in a longitudinal axis direction; and a pressing and driving device that intermittently presses the inner surface near a rear end surface in the axial direction of the double flexible bag member, and turns outward and advances a distal end surface of the double flexible bag member, wherein the medical instrument can be inserted into the body cavity using the longitudinal axis direction of the double flexible bag member as a guide.

With such a configuration, the pressing and driving device intermittently presses the inner surface near the rear end surface in the axial direction of the double flexible bag member, and turns outward and advances the distal end surface of the double flexible bag member. Thus, the distal end surface is intermittently advanced, and releasing a pressing force of the pressing and driving device reduces pressure of the fluid to facilitate bending of the double flexible bag member. Thus, for example, when intermittent pressing near the rear end surface of the double flexible bag member by the pressing and driving device causes the distal end surface of the double flexible bag member to advance and be brought into contact with a wall surface of a bent part of the body cavity, the double flexible bag member receives a reaction force from the wall surface, because the distal end surface presses the inner wall of the body cavity when the pressing and driving device presses the rear end surface of the double flexible bag member. Then, releasing pressing of the rear end surface of the double flexible bag member by the pressing and driving device reduces internal pressure of the fluid, and the double flexible bag member is bent in a bending direction of the body cavity by the reaction force from the wall surface in the body cavity. Thus, the pressing and driving device intermittently presses the inner surface near the rear end surface in the axial direction of the double flexible bag member, thereby allowing the double flexible bag member to be easily advanced along an inner shape of the body cavity without grasping the inner shape, and providing good operability.

After the double flexible bag member is inserted into the body cavity, the insertion portion of the medical instrument can be inserted while being guided along the double flexible bag member, thereby allowing the insertion portion of the medical instrument to be easily inserted. Further, the insertion portion of the medical instrument is not restricted by the double flexible bag member, and thus the distal end can be relatively freely moved to provide satisfactory operability of the medical instrument.

The above and other objects, features and advantages of the invention will become more clearly understood from the following description referring to the accompanying drawings.

### BRIEF DESCRIPTION OF THE DRAWINGS

FIGS. 1 to 24 show a first embodiment of the present invention,

FIG. 1 shows an entire configuration of an endoscope system according to the first embodiment;

FIG. 2 is a perspective view of a pressure increasing and reducing pump device;

FIG. 3 illustrates an insertion state of an insertion guide system;

FIG. 4 is a perspective view of a guide sheath unit during conveyance;

FIGS. 5A to 5D show a production process of a guide sheath,

FIG. 5A illustrates a process of forming a cylindrical member;

FIG. 5B illustrates a process of turning the cylindrical member outward;

FIG. 5C illustrates a process of joining opposite ends of the cylindrical member;

FIG. 5D illustrates a process of moving a secured portion of the guide sheath toward an inner periphery;

FIG. 6 is an enlarged sectional view of essential portions of the guide sheath unit;

FIG. 7 is a perspective view of the guide sheath unit with a gas being supplied to the guide sheath;

FIG. 8 is a sectional view taken along the line VIII-VIII in FIG. 7;

FIG. 9 is a sectional view taken along the line IX-IX in FIG. 8;

FIG. 10 is a perspective view of a pressing member;

FIG. 11 is a perspective view showing an operation of the guide sheath using the pressing member;

FIG. 12A is a sectional view corresponding to FIG. 8 showing a state where a pressing force of the pressing member to the guide sheath is released;

FIG. 12B is a sectional view corresponding to FIG. 8 showing a state where the pressing member is pressed into a rear end of the guide sheath;

FIG. 12C a sectional view corresponding to FIG. 8 showing a state where the pressing force of the pressing member to the rear end of the guide sheath is released;

FIG. 13 illustrates a state where a grip of a guide sheath unit is fitted to an anus;

FIG. 14 illustrates a state where the guide sheath is advanced to a rectum;

FIG. 15 illustrates a state where a distal end of the guide sheath presses up an intestinal wall;

FIG. 16 illustrates a state where the distal end of the guide sheath is bent;

FIG. 17 illustrates a state where the distal end of the guide sheath reaches a cecum;

FIG. 18 illustrates a state where an insertion portion of an endoscope is inserted into the guide sheath;

FIG. 19 illustrates a state where a gas supplied to the guide sheath is reduced in pressure;

FIG. 20 illustrates a state where the guide sheath and the insertion portion of the endoscope are removed from an intestinal tract;

FIG. 21 is a chart showing states of pressure of the gas to the guide sheath in the respective states;

FIG. 22 illustrates a state where the guide sheath and the insertion portion of the endoscope are removed from the intestinal tract of a subject;

FIG. 23 illustrates a state where the guide sheath is removed from the insertion portion of the endoscope;

FIG. 24 illustrates a state where an insertion portion of an endoscope is inserted along a guide sheath according to another aspect;

FIGS. 25 and 26 snow a second embodiment of the present invention,

FIG. 25 is a half-sectional side view of a guide sheath;

FIG. 26 is a side view of a guide sheath unit;

FIG. 27 is a side view of a guide sheath unit according to a third embodiment;

FIGS. 28 to 31 show a fourth embodiment of the present invention,

FIG. 28 is a perspective view of a pressing and driving device;

FIG. 29 is an exploded perspective view of the pressing and driving device;

FIGS. 30A and 30B are sectional views taken along the line XXX-XXX in FIG. 28,

FIG. 30A illustrates a state where a pressing member is retracted;

FIG. 30B illustrates a state where the pressing member is protruded; and

FIG. 31 is a perspective view showing a use mode of the pressing and driving device.

### DETAILED DESCRIPTION OF THE PREFERRED EMBODIMENT

Now, an embodiment of the present invention will be described with reference to the drawings.

### First Embodiment

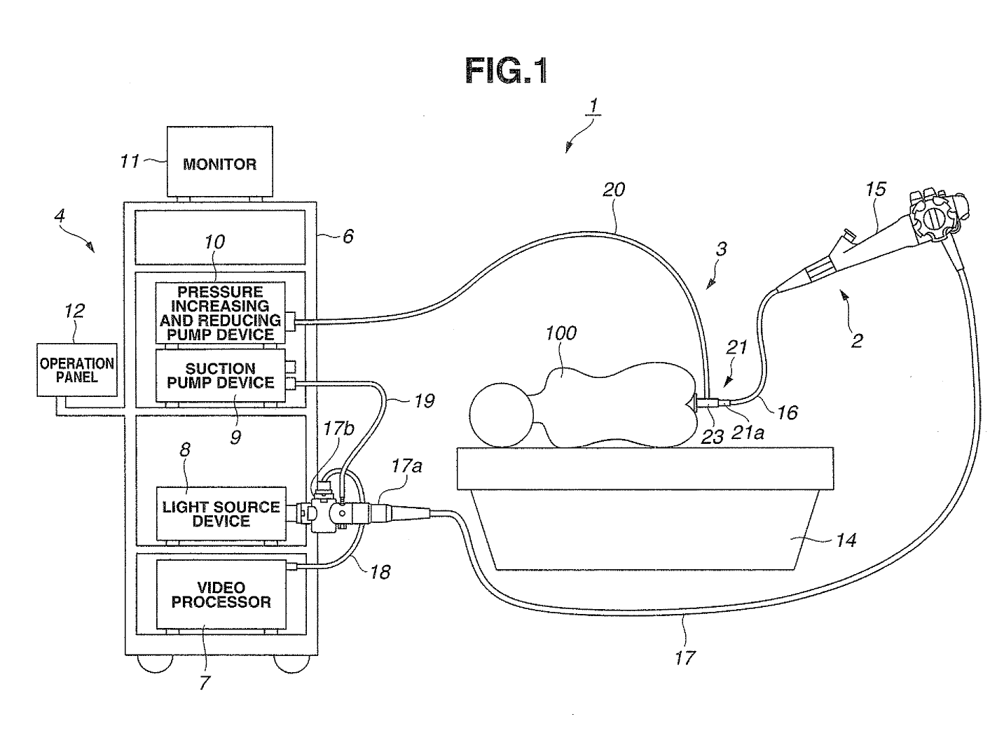

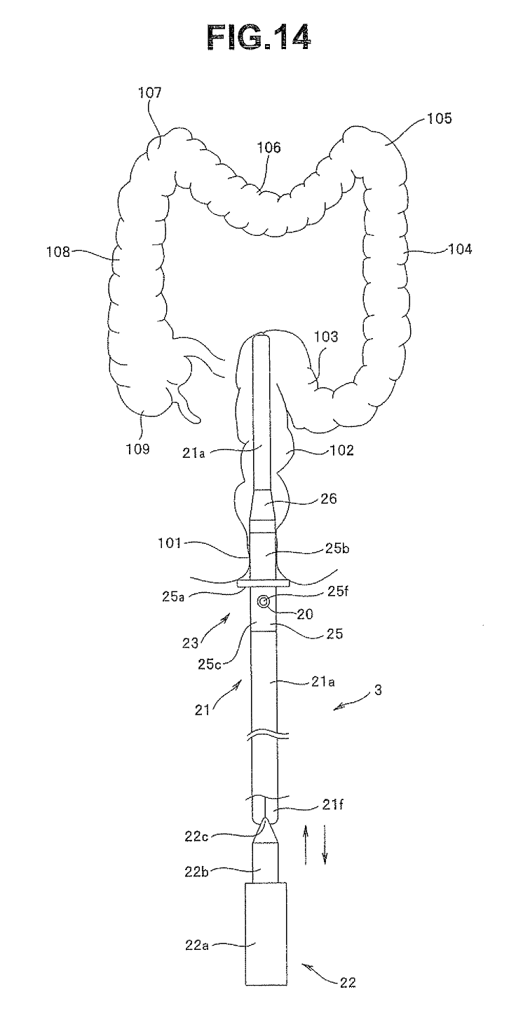

FIGS. 1 to 24 show a first embodiment of the present invention. FIG. 1 shows an entire configuration of an endoscope system. In the present embodiment, a case of performing a large intestine endoscopy using an endoscope system will be exemplified and described. In FIG. 1, an endoscope system 1 includes an endoscope 2 as an example of a medical instrument, an insertion guide system 3 as an example of a medical instrument insertion guide system, and external devices 4 connected to the endoscope 2 and the insertion guide system 3.

The external devices 4 are collectively placed in a trolley 6. In the trolley 6, a video processor 7, a light source device 8, a suction pump device 9, and a pressure increasing and reducing pump device 10 as the external devices are placed in order from a bottom side. A monitor 11 that displays an endoscope image or the like is placed on a top surface of the trolley 6, and an operation panel 12 such as a touch panel or a keyboard is provided on one side of the trolley 6. The pressure increasing and reducing pump device 10 serves as both a pump device and a pressure increasing and reducing device of the present invention.

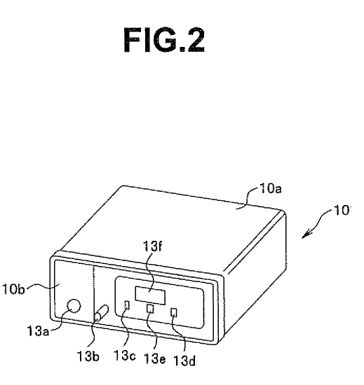

As shown in FIG. 2, the pressure increasing and reducing pump device 10 increases pressure of a fluid (for example, air or carbon dioxide) and supplies the fluid to a guide sheath 21a described later provided in the insertion guide system 3, and deaerates the fluid and reduces the pressure of the fluid. A front panel 10b is mounted to a front surface of a main body 10a of the pressure increasing and reducing pump device 10, a pump body and a system for pressure control of the pump body are included in the pressure increasing and reducing pump device 10, and power supply cables extending from the pump body and the system are provided on a back surface. The power supply cables (not shown) are connected to a power supply to allow electric power to be supplied to the pump body and the system.

On the front panel 10b, a power switch 13a, a pump side tube connecting portion 13b, button switches of a pressure reducing button 13c, a pressure increasing button 13d, and a leak button 13e for setting pump pressure, and a pressure display panel 13f are provided. The buttons 13c, 13d and 13e are provided, for example, on a remote controller to allow remote operation of the pressure increasing and reducing pump device 10.

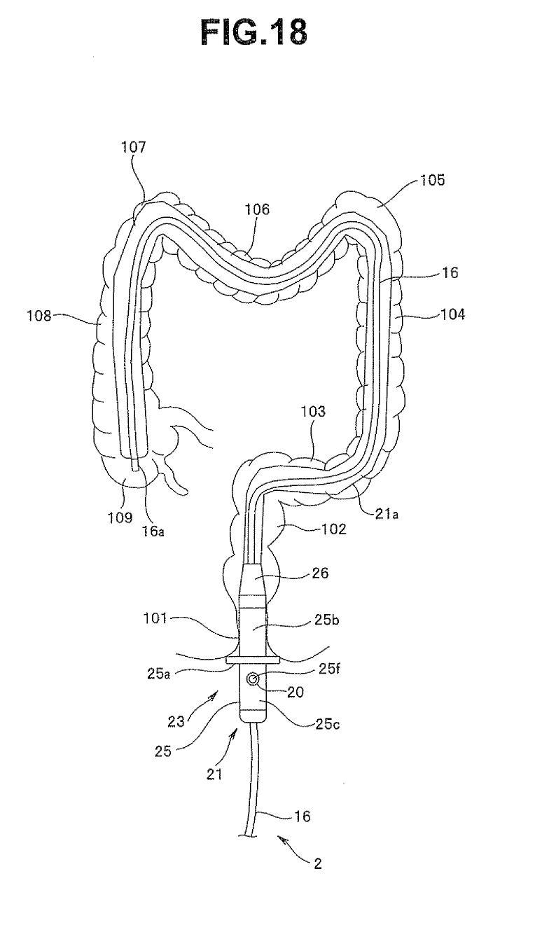

The endoscope 2 includes an operation portion 15 grasped by an operator for an operation, an elongated flexible insertion portion 16 formed on a front end of the operation portion 15 and inserted into a large intestine as a body cavity, and a universal cord 17 having a proximal end extending from a side portion of the operation portion 15. An image pickup device (not shown) such as a CCD for picking up images of an observation area is provided in a distal end portion 16a (see FIG. 18) of the insertion portion 16.

A light source connector 17a is provided on a distal end portion of the universal cord 17. The light source connector 17a is connected to the light source device 8. The light source device 8 includes an illumination light source such as a halogen light source, and an illumination light outputted from the illumination light source is emitted through a light guide fiber (not shown) passed through the universal cord 17 from an illumination window (not shown) provided in the distal end portion of the insertion portion 16 to light a subject. Further, an electric connector 17b is connected to the light source connector 17a, an image pickup cable 18 extending from the electric connector 17b is connected to the video processor 7, and the video processor 7 is connected to the monitor 11. The video processor 7 controls the image pickup device provided in the distal end portion 16a of the insertion portion 16 of the endoscope 2, processes an electric signal of a subject image picked up by the image pickup device, and displays a corresponding image on the monitor 11.

A tube having one end opening in a distal end surface of the distal end portion 16a through the universal cord 17 and the insertion portion 16 of the endoscope 2 has the other end opening in the light source connector 17a, and a suction pump device 9 is connected to the opening via a suction tube 19. The suction pump device 9 is operated to collect tissue or the like of the subject observed by the endoscope 2. Reference numeral 14 denotes an examination table, and reference numeral 100 denotes the subject.

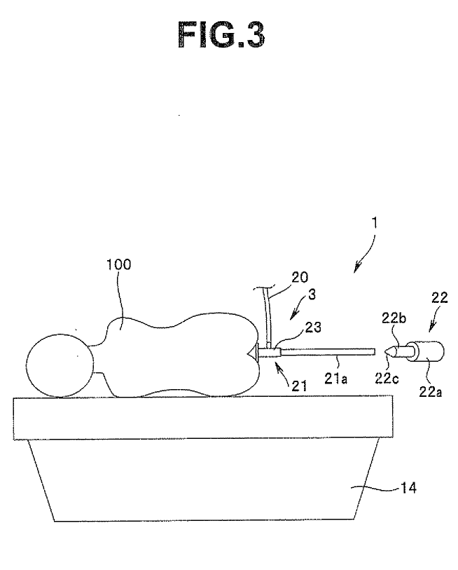

Next, a configuration of the insertion guide system 3 will be described. As shown in FIGS. 1 and 3, the insertion guide system 3 includes a fluid supply and discharge tube 20, a guide sheath unit 21, and a pressing member 22 as a pressing and driving device.

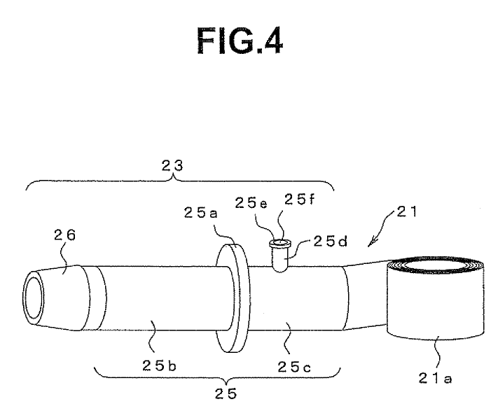

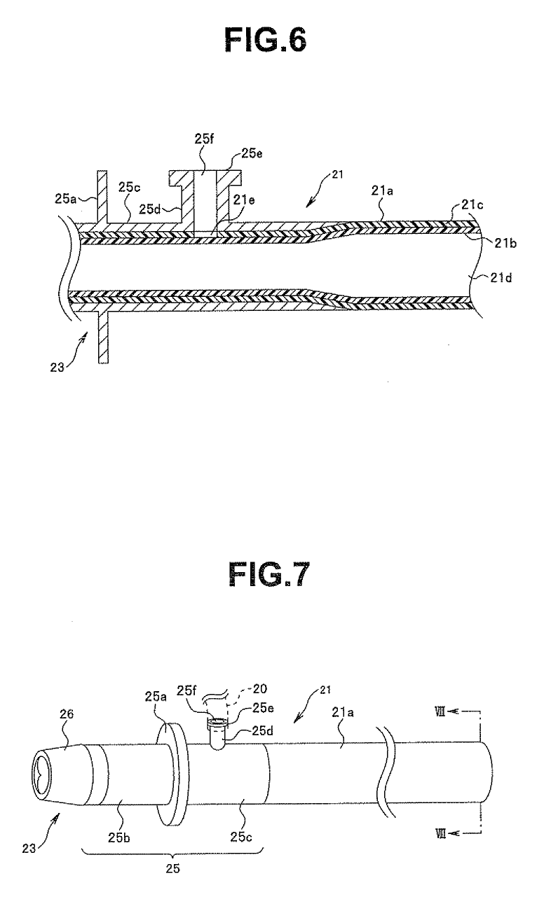

As shown in FIG. 4, the guide sheath unit 21 includes a sheath support member 23, and a guide sheath 21a as a double flexible bag member secured to the sheath support member 23. Further, the sheath support member 23 includes a main body 25, and a guide cap 26 secured to a distal end of the main body 25. The main body 25 is formed of substantially cylindrical rigid resin or an elastic member. A flange 25a is formed on a middle in a longitudinal direction of an outer surface of the main body 25, an insertion portion 25b is formed on a front of the flange 25a, and a grip portion 25c is formed on a back of the flange 25a. Further, a tube connecting portion 25d protrudes radially from an outer surface of the grip portion 25c. A flange portion 25e is formed on an opening end of the tube connecting portion 25d, one end of the fluid supply and discharge tube 20 is mounted to the flange portion 25e, and the other end of the fluid supply and discharge tube 20 is connected to the pump side tube connecting portion 13b of the pressure increasing and reducing pump device 10. In the drawing, the pump side tube connecting portion 13b is shown protruding on a side surface for convenience. As shown in FIG. 6, a passing hole 25f provided in the tube connecting portion 25d opens in an inner wall of the main body 25.

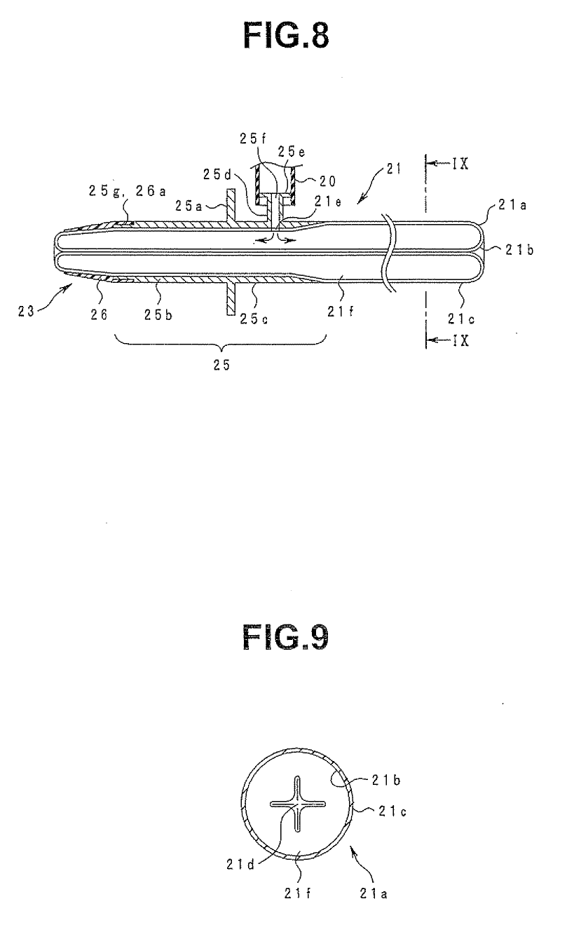

As shown in FIG. 8, a step portion 25g is formed on an outer surface of a distal end portion of the main body 25. A step portion 26a formed on an inner surface of a rear end of the guide cap 26 is fitted on the step portion 25g, and the step portions 25g and 26a are secured by bonding or welding. The guide cap 26 is formed of a cylindrical member of flexible resin or an elastic member having a tapered distal end, and the inner surface of the rear end is continuous with an inner surface of the main body 25.

Further, the guide sheath 21a is passed through the main body 25 and the guide cap 26. The guide sheath 21a is made of flexible material harmless to human body, and formed into a double flexible bag shape having an inner surface 21b and an outer surface 21c continuously formed in a longitudinal axis direction as shown in FIG. 6. An opening 21e coaxial with the passing hole 25f and having substantially the same diameter as the passing hole 25f is provided in the outer surface 21c, which faces the passing hole 25f provided in the tube connecting portion 25d, of the guide sheath 21a secured to an inner periphery of the sheath support member 23a. A gas as an example of a fluid is supplied and discharged through the opening 21e to and from a space 21f formed between the outer surface 21c and the inner surface 21b of the guide sheath 21a. As shown in FIGS. 8 and 9, when the gas is supplied to the space 21f, the inner surface 21b is expanded toward an axial center, and a section of the space 21f perpendicular to a longitudinal direction becomes substantially annular.

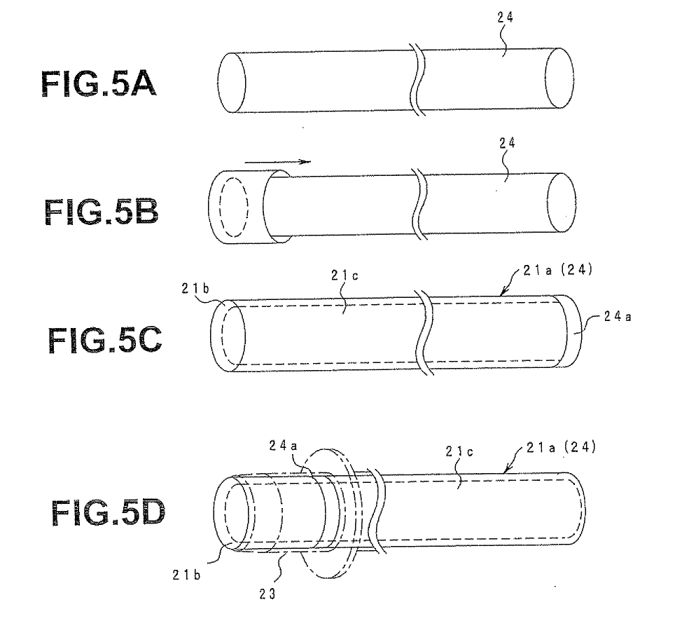

The guide sheath 21a is produced by a process, for example, shown in FIGS. 5A to 5D. Specifically, a cylindrical member 24 is first formed of flexible material harmless to human body and having little stretchability (FIG. 5A). The cylindrical member 24 has an outer diameter smaller than a minimum cavity diameter of a large intestine to be examined, an inner diameter slightly larger than a maximum diameter of the insertion portion 16 provided in the endoscope 2, and a length slightly longer than twice a generally assumed length of a large intestine.

Then, one end of the cylindrical member 24 is turned outward (FIG. 5B) and moved toward the other end, and a joint 24a between the opposite ends is sealed by bonding or welding to form the guide sheath 21a. Then, an inner periphery of the guide sheath 21a forms the inner surface 21b, an outer periphery forms the outer surface 21c, and a passageway 21d through which the insertion portion 16 of the endoscope 2 is passed is formed by the inner surface 21b (FIG. 5C).

Then, the inner surface 211b and the outer surface 21c are axially moved, one end is turned inward and the other end is turned outward, and the joint 24a that is a secured portion of the guide sheath 21a is moved to a position facing an inner periphery of the sheath support member 23 (FIG. 5D). Then, the completed guide sheath 21a is passed through the sheath support member 23, and the outer surface 21c including the joint 24a sealed by bonding or welding is secured to the inner periphery of the sheath support member 23 with a distal end portion of the guide sheath 21a being substantially in alignment with a distal end of the guide cap 26.

The guide sheath unit 21 thus configured is made flat into a strip, rolled, and temporarily secured to the sheath support member 23 with a simple securing tape such as an adhesive tape as shown in FIG. 4 during conveyance.

In use, the other end of the fluid supply and discharge tube 20 having one end mounted to the pump side tube connecting portion 13b provided on the pressure increasing and reducing pump device 10 is connected to the tube connecting portion 25d protruding from the main body 25 of the sheath support member 23. Then, a gas is increased in pressure and supplied from the pressure increasing and reducing pump device 10 through the tube 20 to the tube connecting portion 25d. Then, the gas is supplied to the space 21f in the guide sheath 21a, the pressure of the gas expands the space 21f between the inner surface 21b and the outer surface 21c to extend the guide sheath 21a into a rod shape as shown in FIGS. 7 and 8 and narrow the passageway 21d formed by the inner surface 21b as shown in FIG. 9. The guide sheath 21a is formed by folding back the cylindrical member 24 having little stretchability, and thus even if the guide sheath 21a is expanded by an increase in internal pressure, a maximum diameter is substantially the same as the diameter of the cylindrical member 24. The pressure increasing and reducing pump device 10 includes a valve body that holds the pressure of the gas supplied to the space 21f, and the valve body seals the space 21f.

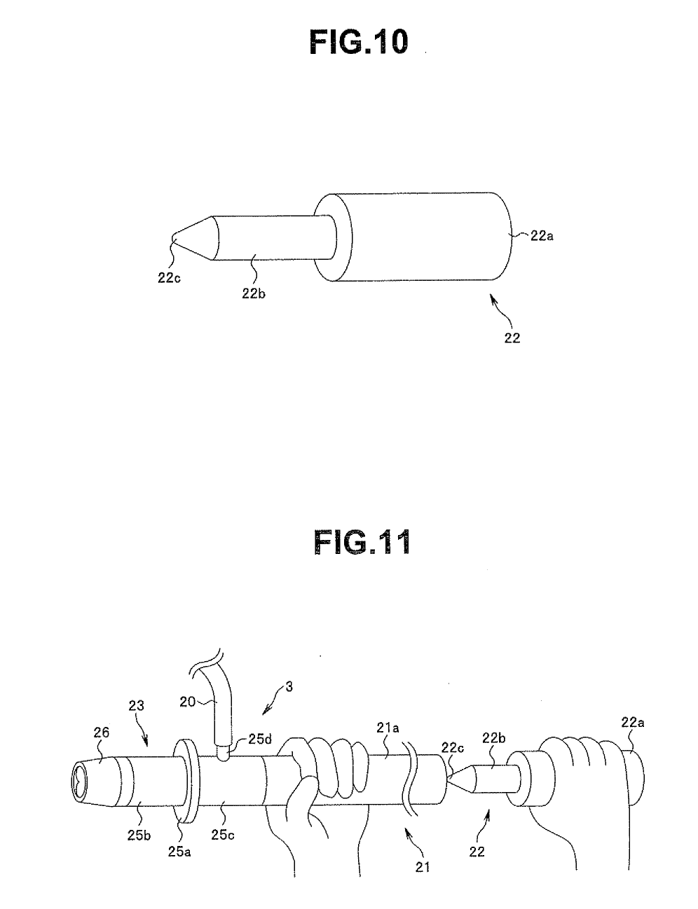

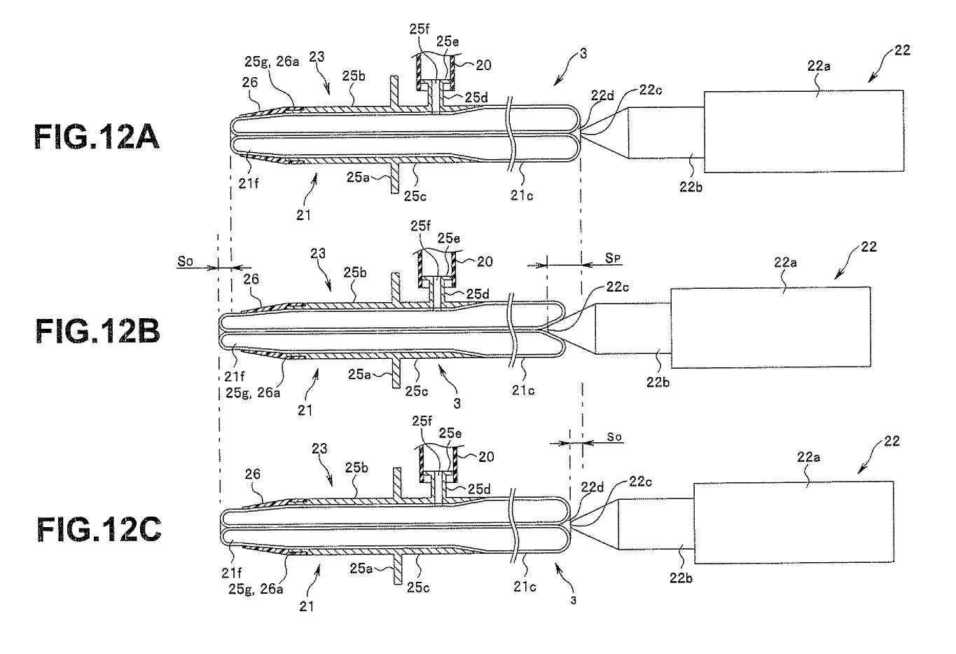

As shown in FIG. 10, the pressing member 22 is made of relatively rigid material, and a pressing portion 22b protrudes from a distal end of a grasping portion 22a. As shown in FIG. 11, the grasping portion 22a is grasped by an operator, and has a length and a thickness for easy grasping. The pressing portion 22b has a substantially cylindrical shape with a substantially conical distal end insertion portion 22c, and has an outer diameter smaller than an outer diameter of the guide sheath 21a. Thus, as shown in FIG. 12B, when the distal end insertion portion 22c of the pressing portion 22b of the pressing member 22 is abutted against the rear end of the guide sheath 21a and then pressed, the entire outer periphery of the insertion portion can be pressed into the inner surface 21b.

Next, an operation will be described in performing large intestine endoscopy by using the endoscope system 1 having such a configuration and inserting per anum the insertion portion 16 provided in the endoscope 2.

First, the other end of the fluid supply and discharge tube 20 having one end connected to the pump side tube connecting portion 13b of the pressure increasing and reducing pump device 10 is connected to the tube connecting portion 25d protruding from the sheath support member 23 of a new guide sheath unit 21 as shown in FIG. 4. Then, the pressure increasing button 13d of the pressure increasing and reducing pump device 10 is turned on. Then, a gas increased in pressure is fed from a pump body (not shown) provided in the pressure increasing and reducing pump device 10 through the fluid supply and discharge tube 20 to the passing hole 25f provided in the tube connecting portion 25d.

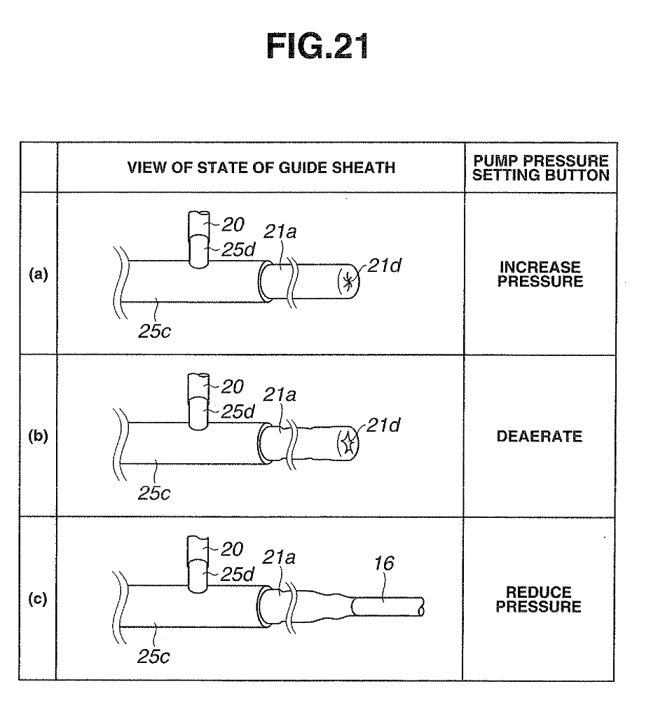

Thus, as shown in FIGS. 6 and 21(a), the gas is supplied through the passing hole 25f from the opening 21e provided in the outer surface 21c of the guide sheath 21a secured to the inner periphery of the sheath support member 23 to the space 21f between the outer surface 21c and the inner surface 21b. Then, as shown in FIGS. 7 to 9, the guide sheath 21a is expanded into an elongated flexible rod shape having a substantially doughnut-shaped section by the pressure of the supplied gas.

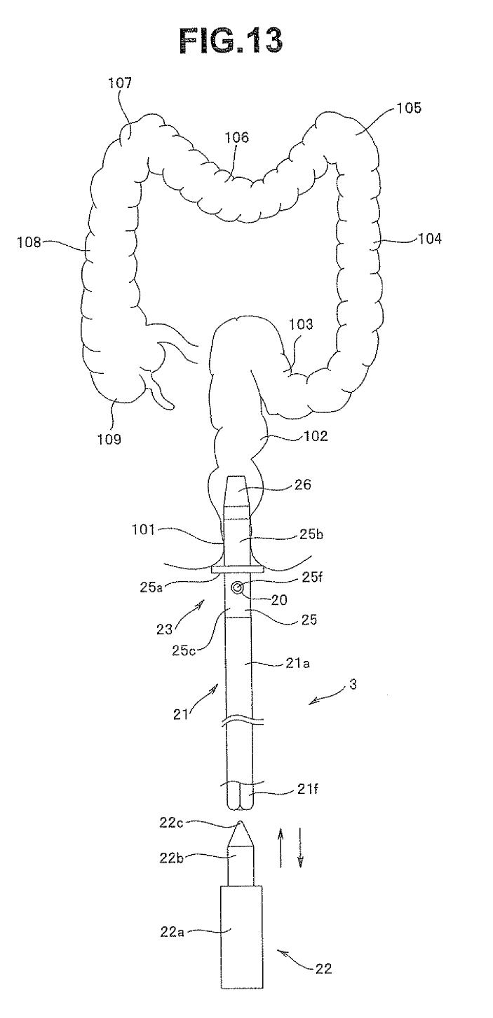

Then, the operator grasps the grip portion 25c of the main body 25 provided in the sheath support member 23 to insert the insertion portion 25b on a distal end side of the main body 25 and the guide cap 26 secured to the insertion portion 25b into an anus 101 of a subject 100 lying on the examination table 14 (see FIGS. 1 and 3) as shown in FIG. 13, and abut the flange 25a against a bottom of the subject 100.

Then, as shown in FIG. 11, the operator grasps the pressing member 22, and presses the distal end insertion portion 22c of the pressing member 22 into a rear end opening of the passageway 21d formed in the guide sheath 21a, for example, by a stroke Sp as shown in FIG. 12B from a state in FIG. 12A. Then, the inner surface 21b of the rear end of the guide sheath 21a is pressed radially outward by the pressing portion 22b of the pressing member 22.

This instantaneously increases pressure in the space 21f of the guide sheath 21a, and the pressure and a propulsion force of the inner surface 21b cause the outer surface 21c of the rear end of the guide sheath 21a to be moved inward toward the inner surface 21b, and at the same time, the inner surface 21b of the distal end surface of the guide sheath 21a is pressed forward, for example, by a stroke So. The guide sheath 21a is secured to the inner periphery of the sheath support member 23, and thus the inner peripheral surface of the guide sheath 21a is moved outward and protruded as indicated by the arrows. At this time, the guide sheath 21a forms a rod shape with relatively high linearity because the pressure in the space 21f is high.

Then, as shown in FIG. 12C, when the pressing force of the pressing member 22 to the guide sheath 21a is released, the rear end of the guide sheath 21a becomes shorter by a stroke So substantially equal to the protrusion stroke So. Thus, the operator intermittently repeatedly presses the distal end insertion portion 22c of the pressing member 22, and thus the distal end surface of the guide sheath 21a is intermittently protruded, and the guide sheath 21a is intermittently advanced from the anus 101 into a deep part of a rectum 102. Then, when the distal end surface of the guide sheath 21a abuts against a deepest part of the rectum 102, the distal end surface is intermittently advanced for some time while extending a back wall of the rectum 102.

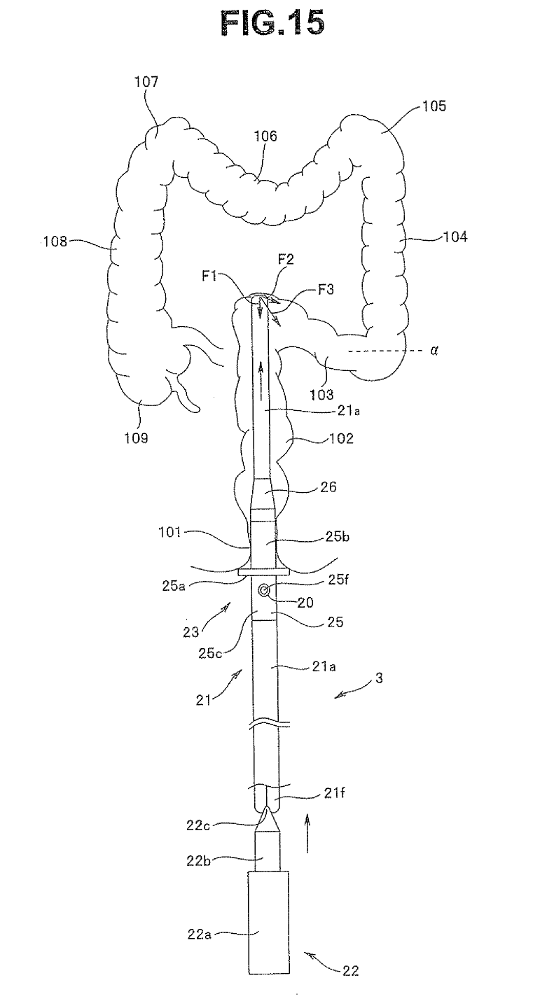

The large intestine is secured at a junction α between a sigmoid colon and a descending colon. Thus, when an intestinal wall of the deep part of the rectum 102 is extended by the distal end surface of the guide sheath 21a, a reaction force F1 from the intestinal wall and a pulling force F2 of the intestine to return apply to the distal end surface of the guide sheath 21a as shown in FIG. 15.

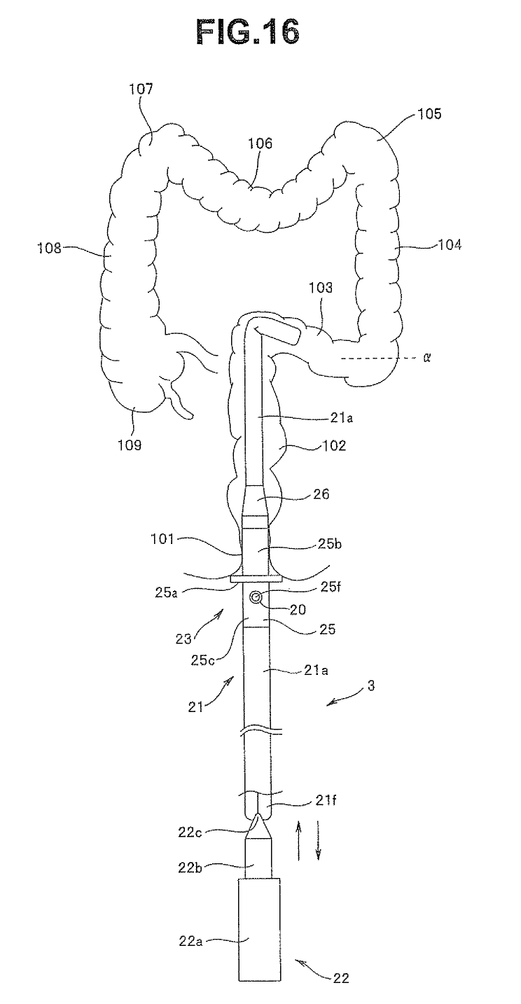

As described above, the distal end surface of the guide sheath 21a is protruded by the increase in the pressure in the space 21f and the forward propulsion force applied to the inner surface 21b when the pressing member 22 is pressed near the rear end surface of the guide sheath 21a. Thus, when the pressing member 22 is released, the pressure in the space 21f and the propulsion force to the inner surface 21b are reduced. Thus, as shown in FIG. 16, the distal end surface of the guide sheath 21a is bent in a direction of a resultant force F3 of the reaction force F1 and the pulling force F2. Thus, the pressure of the gas supplied to the space 21f is set so that the pressure in the space 21f provided in the guide sheath 21a can maintain the rod state of the guide sheath 21a against the pulling force F2 of the intestine to return when the pressing member 22 presses the rear end of the guide sheath 21a in increasing the pressure, and that the guide sheath 21a is bent by the pulling force F2 when the pressing member 22 is removed from the guide sheath 21a. The pressure increasing and reducing pump device 10 includes a safety device. The safety device constantly monitors the pressure in the space 21f and if the pressure during pressing the rear end of the guide sheath 21a with the pressing member 22 exceeds set pressure, the pressure exceeding the set pressure is removed. When the pressure becomes lower than the set pressure, the pressure is adjusted to the set pressure.

Then, when the distal end insertion portion 22c of the pressing member 22 is further pressed into the passageway 21d from the rear end surface of the guide sheath 21a, the inner surface 21b of the distal end is turned outward and advanced. This operation is repeated to propel the distal end surface of the guide sheath 21a.

In the present embodiment, the guide sheath 21a is propelled by turning outward the inner surface 21b of the distal end portion without the guide sheath 21a being moved, and thus the guide sheath 21a is not slid between the distal end surface and the distal end of the guide cap 26 of the sheath support member 23, thereby reducing load on the intestinal wall.

The intermittent pressing operation of the pressing member 22 against the rear end surface of the guide sheath 21a causes the guide sheath 21a to repeatedly enter a rod state with high linearity and a flexible state with bendability. Thus, when the guide sheath 21a is bent and passed through the bent part in the large intestine as shown in FIGS. 15 and 16, the position of the distal end surface of the guide sheath 21a needs not to be always precisely grasped. Specifically, the pressing member 22 is intermittently pressed, and thus the distal end surface that reaches the bent part is naturally bent in the direction of the resultant force F3 by a balance between the reaction force F1 and the pulling force F2. For example, an area into which a contrast medium is injected may be ensured in an axial direction in the space 21f, and the contrast medium may be injected into the area to allow an insertion state of the guide sheath 21a to be easily recognized from outside.

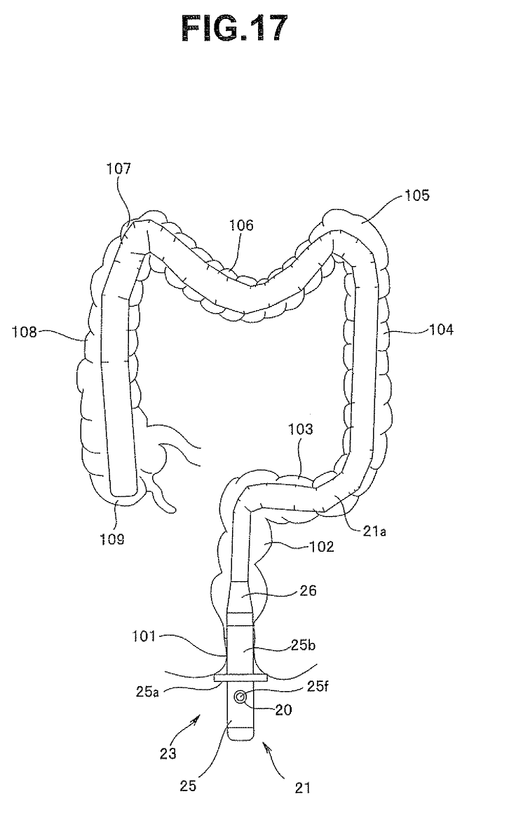

A part extending from the rectum 102 to the sigmoid colon 103 is bent at a relatively sharp angle, but the bent part is passively deformed to have an obtuse angle by the insertion of the guide sheath 21a as shown in FIG. 17. Further, the distal end surface of the guide sheath 21a is turned outward and advanced by the increase in the pressure in the space 21f and the forward propulsion force applied to the inner surface 21b by the pressing force of the pressing member 22. Thus, even a relatively long and flexible guide sheath 21a may be propelled through a descending colon 104, a left colic flexure 105, a transverse colon 106, a right colic flexure 107, and an ascending colon 108 to a cecum 109.

When the distal end surface of the guide sheath 21a reaches the cecum 109, the leak button 13e of the pressure increasing and reducing pump device 10 is pressed. Then, the gas supplied to the space 21 fin the guide sheath 21a is partially deaerated to reduce expansion pressure of the inner surface 21b. Then, as shown in FIG. 21(b), a passing space in the passageway 21d formed by the inner surface 21b is increased to ensure a space through which the insertion portion 16 of the endoscope 2 is passed.

Then, the distal end portion 16a of the insertion portion 16 of the endoscope 2 is passed through the passageway 21d opening in the rear end of the guide sheath 21a and the distal end portion 16a of the insertion portion 16 is guided along the passageway 21d to the cecum 109. At this time, the gas remains in the space 21f in the guide sheath 21a, the pressure of the gas maintains a function of the passageway 21d to guide the insertion portion 16 of the endoscope 2, and each bent part in the large intestine is deformed to have an obtuse angle by the guide sheath 21a as shown in FIG. 17. Thus, the insertion portion 16 of the endoscope 2 can be smoothly guided along the passageway 21d in the guide sheath 21a to the cecum 109.

Then, the distal end portion 16a of the insertion portion 16 of the endoscope 2 reaches the cecum 109, the distal end portion 16a is protruded from the distal end surface of the guide sheath 21a as shown in FIG. 18, and then an intestinal wall of the cecum 109 is first observed. At this time, the distal end portion 16a of the insertion portion 16 is passed through the passageway 21d in the guide sheath 21a in a non-restricted state, and thus can be relatively freely bent. In FIG. 18, the insertion portion of the endoscope inserted into the guide sheath is shown by a solid line for convenience.

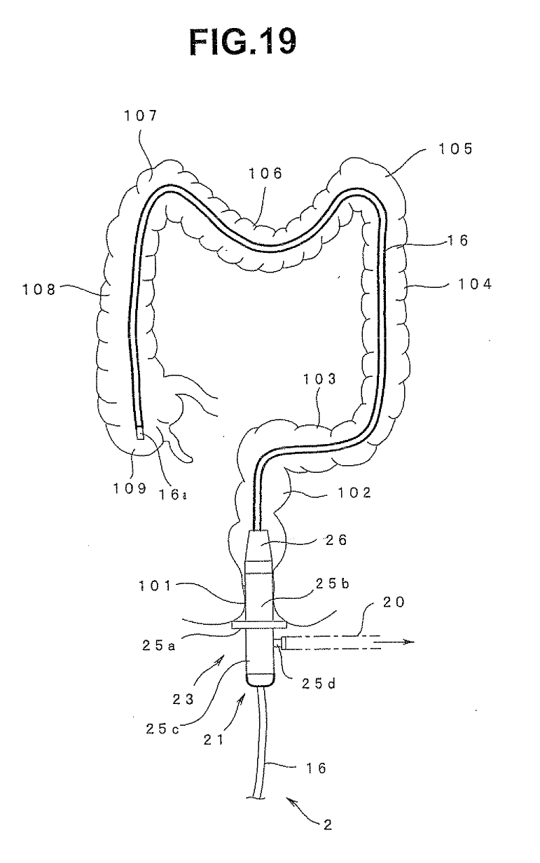

Then, the pressure reducing button 13c of the pressure increasing and reducing pump device 10 is pressed to operate the pressure increasing and reducing pump device 10, suck the gas supplied to the space 21f in the guide sheath 21a, and reduce the pressure in the space 21f. Then, as shown in FIGS. 21(c) and 19, the guide sheath 21a is contracted to increase a space between the inner wall of the intestinal tract and the outer surface 21c of the guide sheath 21a.

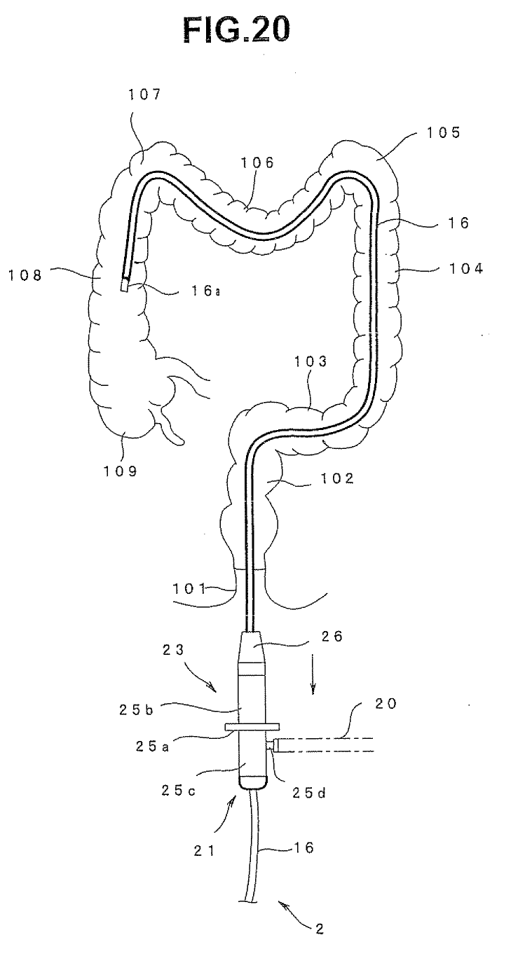

Then, as shown in FIG. 20, the endoscope 2 and the guide sheath 21a are removed from the large intestine, and during the removal, the inside of the intestinal tract is observed. At this time, the space 21f in the guide sheath 21a is reduced in pressure to reduce the entire size of the guide sheath 21a, thus the space is formed between the guide sheath 21a and the inner wall of the intestinal tract, and the guide sheath 21a and the insertion portion 16 of the endoscope 2 can be smoothly removed from the intestinal tract.

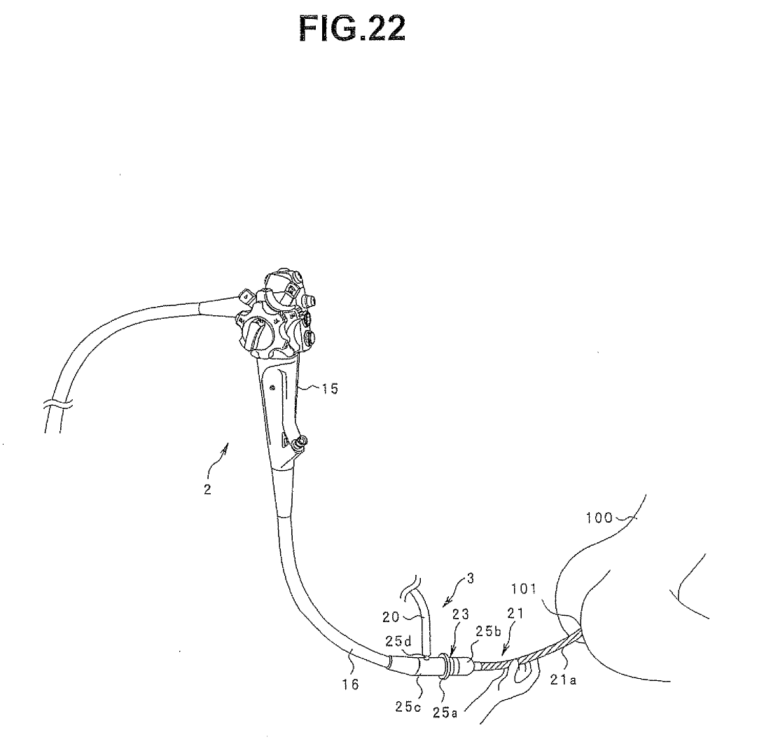

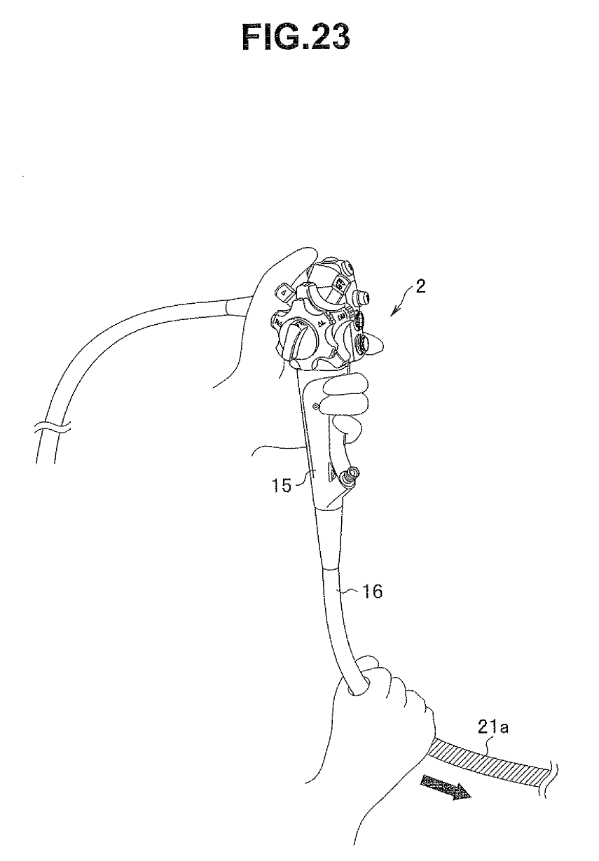

Then, as shown in FIG. 22, the guide sheath 21a and the insertion portion 16 of the endoscope 2 are together removed from the anus 101 to the outside of the body. After the entire removal, the guide sheath 21a is removed from the insertion portion 16 of the endoscope 2 as shown in FIG. 23 and disposed of, and the endoscopy is finished.

As such, the guide sheath 21a provided in the guide sheath unit 21 according to the present embodiment is formed into the elongated flexible rod shape having a doughnut-shaped section by forming the space 21f between the inner surface 21b and the outer surface 21c and supplying the gas at predetermined pressure to the space 21f. The distal end insertion portion 22c of the pressing member 22 is pressed into the passageway 21d opening in the rear end of the guide sheath 21a, and thus the guide sheath 21a enters a rod state with high linearity, and the inner surface 21b of the distal end is turned outward and advanced forward. Thus, as compared with the case where the entire guide sheath 21a is slid in the intestinal tract and advanced, load on the intestinal wall can be reduced and the guide sheath 21a can be easily linearly advanced.

The pressing member 22 intermittently presses the rear end surface of the guide sheath 21a to advance the distal end surface. Thus, from the state where the pressing member 22 presses the rear end of the guide sheath 21a to linearly advance the guide sheath 21a, the pressing force of the pressing member 22 is released to cause the guide sheath 21a to enter a flexible state, and then the distal end surface of the guide sheath 21a is bent in a return direction of the large intestine by the pulling force of the large intestine, thereby allowing the guide sheath 21a to be easily bent and advanced in the bent part in the large intestine.

Thus, the operator can advance the distal end surface along the shape of the intestinal tract simply by intermittently pressing the rear end surface of the guide sheath 21a with the pressing member 22. Thus, there is no need to grasp the shape of the large intestine, and satisfactory operability can be provided. When the insertion portion 16 of the endoscope 2 is inserted into the passageway 21d formed by the inner surface 21b of the guide sheath 21a, the gas supplied to the space 21f is deaerated to slightly extend the passageway 21d formed by the inner surface 21b, and thus the insertion portion 16 of the endoscope 2 can be easily guided to the cecum 109 through the passageway 21d.

Further, the insertion portion 16 of the endoscope 2 is relatively movably passed through the passageway 21d formed in the guide sheath 21a, and thus as shown in FIG. 18, the insertion portion 16 can be relatively freely bent in vertical and lateral directions with the distal end portion 16a being protruded from the guide sheath 21a. This provides satisfactory operability of the endoscope 2, and allows efficient observation of the inside of the intestinal tract.

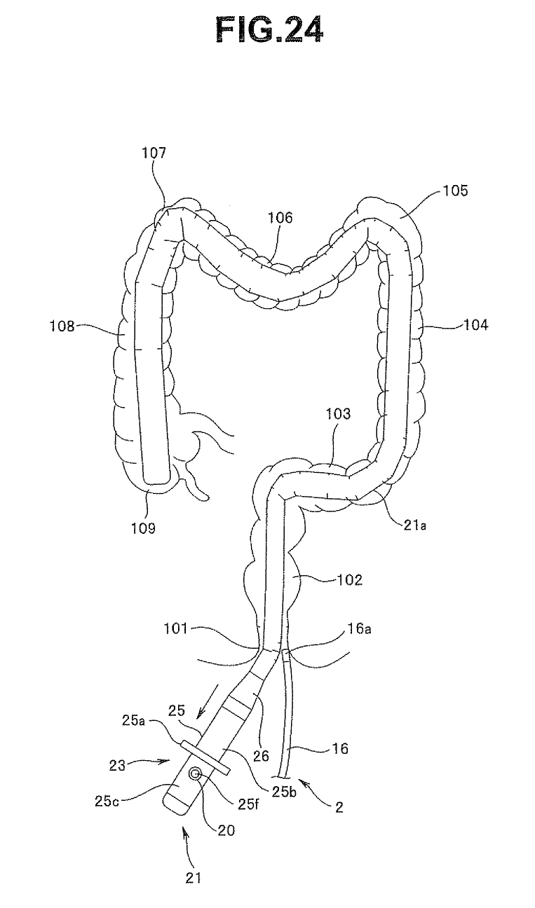

In the above description, the insertion portion 16 of the endoscope 2 is inserted into the passageway 21d formed in the guide sheath 21a and guided to the cecum 109, but as shown in FIG. 24, the insertion portion 16 of the endoscope 2 may be guided along the outer surface 21c of the guide sheath 21a.

Specifically, after the distal end surface of the guide sheath 21a is advanced to the cecum 109, the guide cap 26 and the insertion portion 25b of the main body 25 in the sheath support member 23 are removed from the anus 101 of the subject 100, and the insertion portion 16 of the endoscope 2 is inserted into the outer surface 21c of the guide sheath 21a and the anus 101. Each of the bent parts of the large intestine is deformed to have an obtuse angle by the guide sheath 21a, and thus even outside the guide sheath 21a, the intestinal tract is not excessively extended when the insertion portion 16 of the endoscope 2 passes through the bent part, thereby allowing the insertion portion 16 to be relatively easily advanced. In this case, the gas supplied to the space 21f in the guide sheath 21a may be partially deaerated as described above.

According to this aspect, the insertion portion 16 of the endoscope 2 is not passed through the guide sheath 21a, and thus the insertion portion 16 can be freely moved forward and backward with the distal end surface of the guide sheath 21a being passed in the cecum 109. Thus, an observation area to be noted can be observed simply by forward and backward movement of the insertion portion 16 of the endoscope 2 without inserting and removing the guide sheath 21a, thereby providing ease of handling.

### Second Embodiment

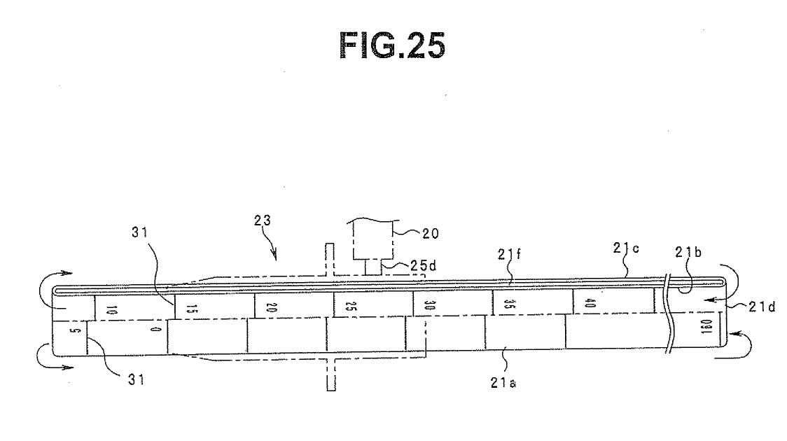

FIGS. 25 and 26 show a second embodiment of the present invention. FIG. 25 is a half-sectional side view of a guide sheath, and FIG. 26 is a side view of a guide sheath unit 21. The same components as in the first embodiment are denoted by the same reference numerals and descriptions thereof will be omitted.

In the present embodiment, the guide sheath 21a described in the first embodiment includes indexes indicating an insertion length.

Specifically, the guide sheath 21a is secured to the inner periphery of the sheath support member 23, and as shown in FIG. 26, when the rear end is pressed by the distal end insertion portion 22c of the pressing member 22 (see FIG. 11), the outer surface 21c of the rear end is turned inward and the inner surface 21b of the distal end surface is turned outward, and thus the distal end surface is advanced. In the present embodiment, indexes 31 (scales in the drawings) are indicated at predetermined intervals on the inner surface 21b and the outer surface 21c of the guide sheath 21a with reference to the distal end surface of the guide cap 26 provided in the sheath support member 23.

The operator can recognize the indexes 31 when feeding the outer surface 21c of the rear end of the guide sheath 21a toward the inner surface 21b using the pressing member 22, and grasp a substantial position of the distal end surface of the guide sheath 21a. When inserting the distal end portion 16a of the insertion portion 16 of the endoscope 2 along the passageway 21d or the outer surface 21c of the guide sheath 21a, the operator can recognize the indexes 31 with an endoscope image, and grasp a substantial insertion position. The shown indexes 31 are examples, and for example, the indexes may be bar codes, and the video processor 7 may recognize bar code data whose images are picked up by the endoscope 2; and an insertion depth of the distal end insertion portion 22c from the anus 101 indicated by the bar code data may be displayed on the monitor 11.

### Third Embodiment

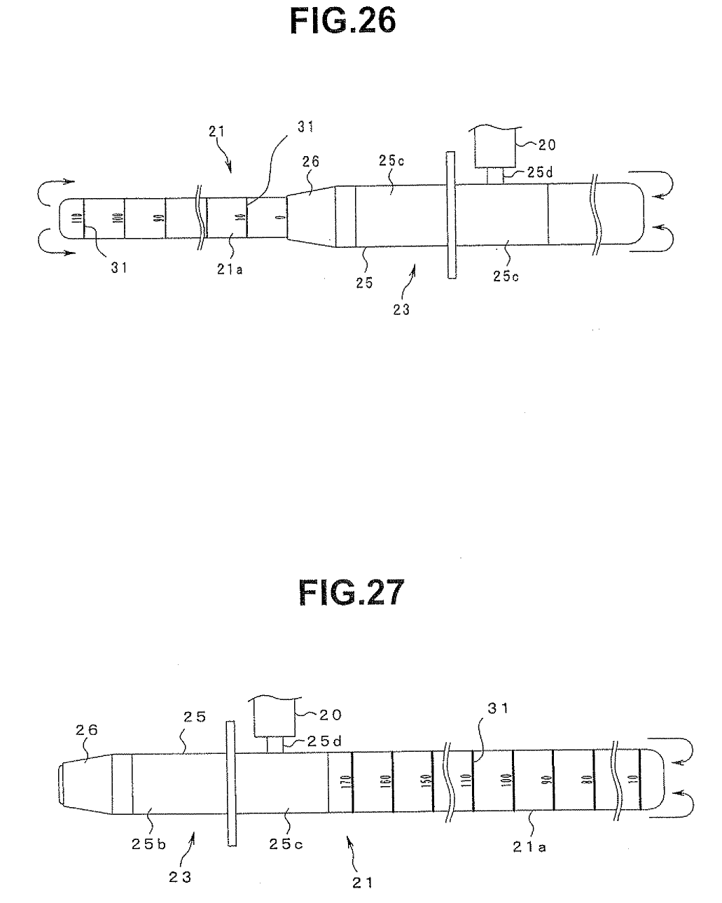

FIG. 27 is a side view of a guide sheath unit according to a third embodiment of the present invention. The present embodiment is a variant of the second embodiment. In the second embodiment, the indexes 31 indicating the insertion length of the guide sheath 21a are set with reference to the distal end surface of the guide cap 26 provided in the sheath support member 23, but in the present embodiment, the insertion length of the guide sheath 21a can be grasped at a hand side of the operator.

Specifically, as shown in FIG. 27, in an initial state where the distal end of the guide sheath 21a is in alignment with the distal end of the guide cap 26, the rear end of the guide sheath 21a at a hand side of the operator (a right side in FIG. 27) indicates 0 [cm], and an index 31 (170 [cm] in FIG. 27) indicating a maximum insertion length of the guide sheath 21a is provided on the rear end of the grip portion 25c of the main body 25 in the sheath support member 23.

Thus, the operator can recognize the index 31 indicated on the rear end of the guide sheath 21a while feeding the guide sheath 21a into the large intestine of the subject 100, and can easily grasp the insertion length of the guide sheath 21a.

### Fourth Embodiment

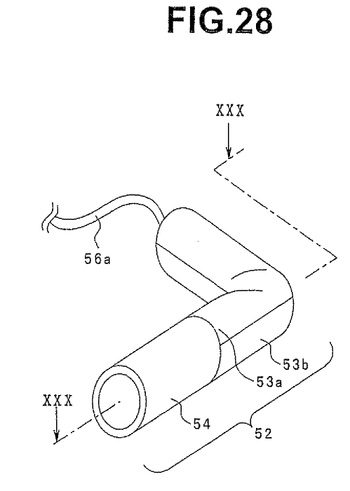

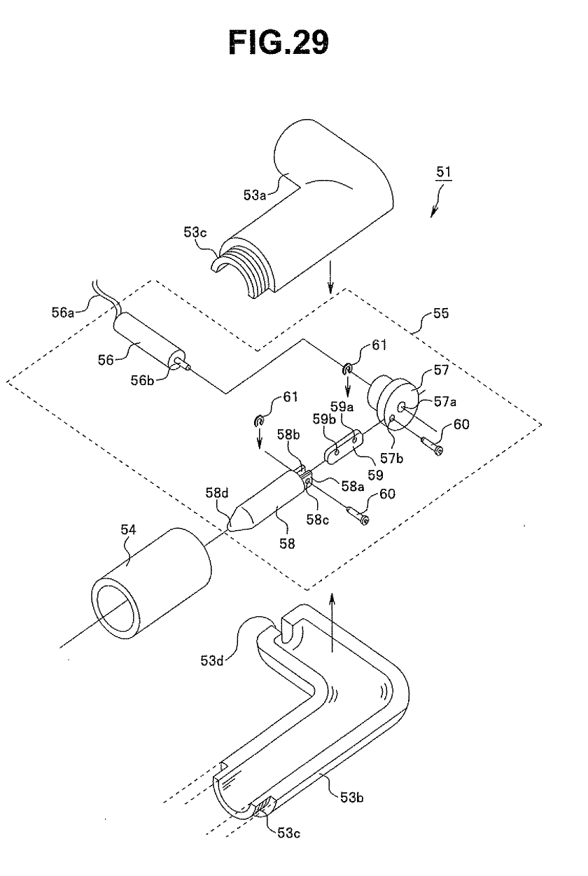

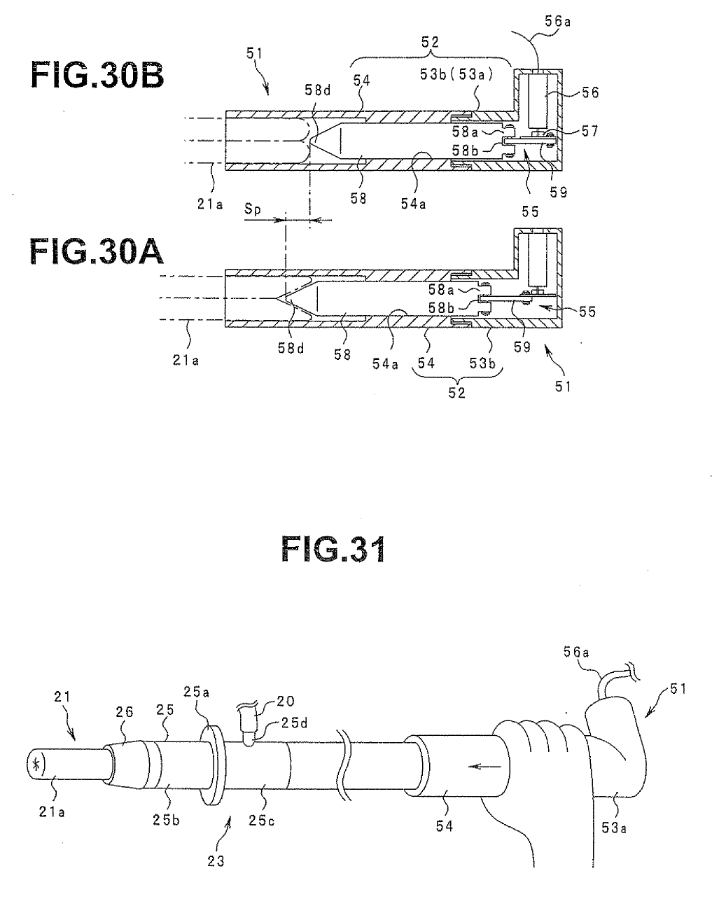

FIGS. 28 to 31 show a fourth embodiment of the present invention. FIG. 28 is a perspective view of a pressing and driving device, FIG. 29 is an exploded perspective view thereof, FIGS. 30A and 30B are sectional views taken along the line XXX-XXX in FIG. 28, and FIG. 31 is a perspective view showing a use mode of the pressing and driving device. The configuration of the guide sheath unit 21 is the same as in the first embodiment, and the description thereof will be omitted.

In the first embodiment, the distal end insertion portion 22c of the pressing member 22 is pressed into the rear end of the guide sheath 21a by the pressing force of the operator, but in the present embodiment, the distal end insertion portion 22c of the pressing member 22 is electrically pressed.

Specifically, as shown in FIGS. 28 and 29, a case body 52 of a pressing and driving device 51 includes an upper cover 53a, a lower cover 53b, and a distal end cylinder 54. The covers 53a and 53b have symmetrical shapes and are bonded together in face-to-face relationship. Threaded portions 53c are formed in end outer surfaces of the covers 53a and 53b, and a rear portion of the distal end cylinder 54 is threaded on the threaded portions 53c.

A driving portion 55 is housed in the case body 52. The driving portion 55 includes a motor 56, and a power supply cable 56a connected to an external power supply extends from a rear end of the motor 56. The motor 56 is secured to one of the covers 53a and 53b by a securing member such as a screw, and a shaft hole 57a provided in a center of rotation of a rotor plate 57 is fitted and secured on a spindle 56b protruding from a distal end of the motor 56. The rotor plate 57 has a cylindrical shape having a flange portion at a distal end, and a passing hole 57b is provided in an outer edge of the flange portion. The rotor plate 57 is made of metal material or resin material having high slidability.

The rotor plate 57 and a pressing member 58 are connected by a linkage 59. The linkage 59 is made of metal material or resin material having high slidability and formed into a flat shape, and has support holes 59a and 59b in opposite ends. A pin shaft 60 is passed through one of the support holes 59a and the shaft hole 57a provided in the rotor plate 57. The pin shaft 60 has a flange portion at one end and a ring groove at the other end, and a C-ring 61 is mounted to the ring groove for retaining. The motor 56, the rotor plate 57, and the linkage 59 are components of a driving member of the present invention.

The pressing member 58 is made of metal material or resin material having high slidability, and has a double sided portion 58a protruding on a rear end. The double sided portion 58a has a slit portion 58b and a through hole 58c. The other end of the linkage 59 is passed through the slit portion 58b, a pin shaft 60 is passed through the support hole 59b provided in the other end and the through hole 58c provided in the double sided portion 58a, and a C-ring 61 is mounted to a ring groove formed in the other end of the pin shaft for retaining. The linkage 59 is rotatably supported via the pin shafts 60 by the rotor plate 57 and the double sided portion 58a of the pressing member 58. Further, a distal end insertion portion 58d of the pressing member 58 has a substantially conical shape.

The power supply cable 56a extending from the rear end of the motor 56 extends through a cable groove 53d formed in the lower cover 53b to the outside of the case body 52, and is connected to the power supply. The pressing and driving device 51 includes a power switch though not shown. The power switch is, for example, a momentary switch provided on the case body 52, and turned on by a grasping force of an operator only while the operator is grasping the case body 52. The power switch may be provided on a foot switch instead of the case body 52. The power switch is provided on the footswitch, thereby allowing the operator to operate the motor 56 at any timing.

As shown in FIGS. 30A and 30B, a guide portion 54a having a decreasing diameter is formed in the inner surface of the distal end cylinder 54, and an outer periphery of the pressing member 58 is axially movably supported by the guide portion 54a. When the spindle 56b of the motor 56 rotates, the pressing member 58 linearly reciprocates while being supported by the guide portion 54a via the rotor plate 57 and the linkage 59. The inner periphery of the distal end of the distal end cylinder 54 has a diameter that can be mounted to the outer surface 21c of the guide sheath 21a of the guide sheath unit 21.

In such a configuration, as shown in FIG. 31, the operator grasps the case body 52 of the pressing and driving device 51, the outer surface 21c of the rear end of the guide sheath 21a provided in the guide sheath unit 21 is mounted to the inner periphery of the distal end cylinder 54 of the case body 52, the distal end insertion portion 58d of the pressing member 58 is pressed against the rear end of the guide sheath 21a, and then an unshown power switch is turned on.

Then, the spindle 56b of the motor 56 rotates, and the rotor plate 57 journaled on the spindle 56b rotates. Then, torque of the rotor plate 57 is transmitted to the pressing member 58 by the linkage 59. The pressing member 58 is axially movably supported by the guide portion 54a formed in the inner periphery of the distal end cylinder 54. Thus, when the spindle 56b of the motor 56 rotates, the pressing member 58 linearly reciprocates along the guide portion 54a. As shown in FIGS. 30A and 30B, a stroke Sp of the pressing member 58 during the linear reciprocation is twice a distance between a center of the shaft hole 57a provided in the rotor plate 57 and a center of the passing hole 57b. Thus, the distance between the shaft hole 57a and the passing hole 57b can be set to freely set the stroke Sp.

As shown in FIG. 30A, in a state where the pressing member 58 is retracted, the pressing force to the gas supplied to the space 21f in the guide sheath 21a is released, and thus the guide sheath 21a enters a flexible state. On the other hand, as shown in FIG. 30B, in a state where the pressing member 58 protrudes, the distal end insertion portion 58d is pressed into the rear end of the guide sheath 21a to increase the pressing force to the gas supplied to the space 21f, and the inner surface 21b of the distal end is turned outward and advanced. Thus, the rotation of the motor 56 causes the pressing member 58 to linearly reciprocate, and thus the distal end of the guide sheath 21a is intermittently advanced.

In the present embodiment, the pressing member 58 is electrically linearly reciprocated, and the guide sheath 21a is advanced by the intermittent pressing force at the time. This allows automatic application of the pressing force to the rear end surface of the guide sheath 21a, and reduces load on the operator even in a relatively long-hour operation with convenience.

Having described the preferred embodiments of the invention referring to the accompanying drawings, it should be understood that the present invention is not limited to those precise embodiments and that various changes and modifications thereof could be made by one skilled in the art without departing from the spirit or scope of the invention as defined in the appended claims.

What is claimed is:

1. A medical instrument insertion guide system comprising:
a medical instrument including a long insertion portion to be inserted into a body cavity;
a double flexible bag member that is an annular member made of flexible material and supplied with a fluid and then sealed, and includes an inner surface and an outer surface to be continuously moved in a longitudinal axis direction; and
a pressing and driving device that intermittently presses the inner surface near a rear end surface in the axial direction of the double flexible bag member, and turns outward and advances a distal end surface of the double flexible bag member,
wherein the medical instrument is inserted into the body cavity using the longitudinal axis direction of the double flexible bag member as a guide.

2. The medical instrument insertion guide system according to claim 1, wherein the pressing and driving device includes:
a pressing member having a distal end insertion portion that can abut against the rear end surface of the double flexible bag member and can be linearly moved; and
a driving member that reciprocates linearly the pressing member.

3. The medical instrument insertion guide system according to claim 1 further comprising:
an opening provided in the double flexible bag member for supplying and discharging a fluid; and
a pump device connected to the opening via a fluid tube for supplying and discharging the fluid to and from the double flexible bag member.

4. The medical instrument insertion guide system according to claim 1, wherein the insertion portion of the medical instrument is inserted along the outer surface of the double flexible bag member inserted into the body cavity.

5. The medical instrument insertion guide system according to claim 2, wherein the insertion portion of the medical instrument is inserted along the outer surface of the double flexible bag member inserted into the body cavity.

6. The medical instrument insertion guide system according to claim 1, wherein the insertion portion of the medical instrument is inserted through a passageway formed in the inner surface of the double flexible bag member inserted into the body cavity.

7. The medical instrument insertion guide system according to claim 2, wherein the insertion portion of the medical instrument is inserted through a passageway formed in the inner surface of the double flexible bag member inserted into the body cavity.

8. The medical instrument insertion guide system according to claim 1, wherein the pressing and driving device presses the outer surface near the rear end surface of the double flexible bag member toward the inner surface.

9. The medical instrument insertion guide system according to claim 1, further comprising a pressure increasing and reducing device that increases and reduces pressure of the fluid sealed in the double flexible bag member,
wherein the pressure increasing and reducing device reduces the pressure of the fluid when the insertion portion of the medical instrument is inserted via the double flexible bag member.

10. The medical instrument insertion guide system according to claim 2, further comprising a pressure increasing and reducing device that increases and reduces pressure of the fluid sealed in the double flexible bag members
wherein the pressure increasing and reducing device reduces the pressure of the fluid when the insertion portion of the medical instrument is inserted via the double flexible bag member.

11. The medical instrument insertion guide system according to claim 3, further comprising a pressure increasing and reducing device that increases and reduces pressure of the fluid sealed in the double flexible bag member,
wherein the pressure increasing and reducing device reduces the pressure of the fluid when the insertion portion of the medical instrument is inserted via the double flexible bag member.

12. The medical instrument insertion guide system according to claim 4, further comprising a pressure increasing and reducing device that increases and reduces pressure of the fluid sealed in the double flexible bag member,
wherein the pressure increasing and reducing device reduces the pressure of the fluid when the insertion portion of the medical instrument is inserted via the double flexible bag member.

13. The medical instrument insertion guide system according to claim 5, further comprising a pressure increasing and reducing device that increases and reduces pressure of the fluid sealed in the double flexible bag member,
wherein the pressure increasing and reducing device reduces the pressure of the fluid when the insertion portion of the medical instrument is inserted via the double flexible bag member.

14. The medical instrument insertion guide system according to claim 6, further comprising a pressure increasing and reducing device that increases and reduces pressure of the fluid sealed in the double flexible bag member,
wherein the pressure increasing and reducing device reduces the pressure of the fluid when the insertion portion of the medical instrument is inserted via the double flexible bag member.

15. The medical instrument insertion guide system according to claim 9, wherein when the insertion portion of the medical instrument inserted along the double flexible bag member is removed from the body cavity, the pressure of the fluid is reduced by the pressure increasing and reducing device, and the double flexible bag member is removed together with the insertion portion.

16. The medical instrument insertion guide system according to claim 10, wherein when the insertion portion of the medical instrument inserted along the double flexible bag member is removed from the body cavity, the pressure of the fluid is reduced by the pressure increasing and reducing device, and the double flexible bag member is removed together with the insertion portion.

17. The medical instrument insertion guide system according to claim 11, wherein when the insertion portion of the medical instrument inserted along the double flexible bag member is removed from the body cavity, the pressure of the fluid is reduced by the pressure increasing and reducing device, and the double flexible bag member is removed together with the insertion portion.

18. The medical instrument insertion guide system according to claim 12, wherein when the insertion portion of the medical instrument inserted along the double flexible bag member is removed from the body cavity, the pressure of the fluid is reduced by the pressure increasing and reducing device, and the double flexible bag member is removed together with the insertion portion.

19. The medical instrument insertion guide system according to claim 13, wherein when the insertion portion of the medical instrument inserted along the double flexible bag member is removed from the body cavity, the pressure of the fluid is reduced by the pressure increasing and reducing device, and the double flexible bag member is removed together with the insertion portion.

20. The medical instrument insertion guide system according to claim 14, wherein when the insertion portion of the medical instrument inserted along the double flexible bag member is removed from the body cavity, the pressure of the fluid is reduced by the pressure increasing and reducing device, and the double flexible bag member is removed together with the insertion portion.
